(19) United States
(12) Patent Application Publication
(10) Pub. No.: US 2023/0079542 A1
(43) Pub. Date: Mar. 16, 2023

(54) APPARATUS FOR USE IN THE PERFORMANCE OF COGNITIVE BEHAVIOUR THERAPY AND METHOD OF PERFORMANCE

(71) Applicant: Third Eye Neurotech Ltd, Morpeth (GB)

(72) Inventors: Jeremy Parr, Newcastle-upon-Tyne Tyne and Wear (GB); Helen McConachie, Newcastle-upon-Tyne Tyne and Wear (GB); Paul Smith, Newcastle-upon-Tyne Tyne and Wear (GB)

(21) Appl. No.: 18/051,451

(22) Filed: Oct. 31, 2022

Related U.S. Application Data
(63) Continuation of application No. 14/888,642, filed on Nov. 2, 2015, now abandoned.

(30) Foreign Application Priority Data
May 1, 2013 (GB) 1307896.9

(51) Int. Cl. A61B 5/00; A61B 5/16; G16H 20/70

(52) U.S. Cl. CPC A61B 5/7445; A61B 5/165; G16H 20/70; A61B 5/11

(57) ABSTRACT

An apparatus for use in the conduct of cognitive behavior therapy comprises a surround vision system, sets of recorded scenes each set relating to the same theme, each set representing a different level of difficulty to the individual receiving therapy in dealing with the theme; a controller for controlling the displaying images of a set on the screens of the surround vision system; and a command device. The command device is adapted to communicate with the controller and is used by a person overseeing the delivery of therapy to the individual or by the individual receiving therapy for controlling the display of scenes on the surround vision apparatus.

### CROSS-REFERENCE TO RELATED APPLICATION(S)

This application is a continuation of U.S. application Ser. No. 14/888,642 filed Nov. 2, 2015 for “Apparatus for Use in the Performance of Cognitive Behaviour Therapy and Method of Performance”, which is the U.S. National Phase Application of PCT/GB2014/051355, filed May 1, 2014, which claims priority to Great Britain Application No. 1307896.9, filed May 1, 2013, the contents of which are incorporated herein by reference in their entirety.

### FIELD OF THE INVENTION

The present invention relates to enhancing the conduct of cognitive behavior therapy and in particular to an apparatus for use in such therapy and a method of performing such therapy.

### BACKGROUND OF THE INVENTION

Cognitive behavior therapy is widely used. In relation to individuals, particularly people with autism spectrum disorder, cognitive behavior therapy may be used to assist individuals in becoming accustomed to activities which would otherwise present significant challenges in their daily lives, in particular to overcoming phobias.

For example, a child may find crossing the road, or speaking to a person in a shop extremely difficult. Another child may have a particular fear, for example, of pigeons which restricts their willingness to go out of the house.

It is well documented that such problems may be treated by gradually increasing the challenge posed to the individual, in the context of helping the individual develop strategies for keeping relaxed and gaining confidence.

A known method of attempting to assist an individual with a problem of the type described above includes a psychologist or therapist accompanying the individual to the environment in which the particular problem occurs and increasing the challenge to the individual by small incremental steps.

The psychologist or therapist attempts to control the challenge posed to the individual in the environment. However, this is very difficult to achieve because the real world the environment is in many was not controllable. For example, in the case of an individual who finds going to the till in a shop challenging, the psychologist or therapist may be able to control certain aspects of the individual's experience, for example which shop is entered, when the person at the till is approached, etc. However, the therapist cannot control other shoppers, or the reaction of the person at the till. To overcome difficulties in arranging graded steps in exposure, the therapist may help the individual to imagine scenes in a graded way. However, for individuals with autism control of imagination may be particularly difficult.

It would be desirable to be able to accustom individuals to situations in a more controlled environment. This would not only allow the individuals to progress more rapidly in overcoming problems, but may also relieve pressure on resources and allow more individuals to be treated.

However, in order for an individual to become accustomed to a situation, the individual must believe that the controlled environment is believable as being representative of the problem. For example, looking at pictures in a book or on a computer screen is unlikely to accustom the individual to the problem sufficiently for the individual to then go out into the real world and face the problem.

Surprisingly, it has been found that by exposing individuals to a progressively increasing challenge within a surround vision environment, individuals may overcome their phobias, with transfer of confidence to real life.

### SUMMARY

According to the invention there is provided an apparatus for use in the performance of cognitive behavior therapy, the apparatus comprising:

a surround vision system comprising at least two screens, a plurality of projectors and at least one processor, the at least two screens substantially enclosing a space for receiving an individual receiving treatment, the plurality of projectors configured to project images onto the said at least two screens such that the said images may be viewed from within the said substantially enclosed space, wherein the processor synchronizes the projection of images onto each screen such that the images are perceived by the viewer as being uninterrupted;

sets of recorded scenes each set relating to a theme, each set representing a different level of difficulty to the individual receiving therapy in dealing with the theme;

a controller for controlling the displaying scenes of a set on the screens of the surround vision system; and

a command device adapted to communicate with the controller and for use by a person overseeing the delivery of therapy to the individual or the individual receiving therapy for controlling the display of scenes on the screens of the surround vision system.

The surround vision system advantageously includes a sound system configured to play and emit sound synchronized with the images shown on the screens of the system.

The apparatus may further include one or more anxiety monitoring devices and the or each anxiety monitoring device is advantageously in communication with the controller. Anxiety monitoring devices may include, but are not limited to: a heart rate monitor, blood pressure sensor, skin temperature sensor, an eye tracker for example.

The command device may comprise a hand held remote control. The command device may be adapted to interact with a screen of the surround vision apparatus.

The command device may comprise a part of an eye tracking system.

The command device may include a glove adapted to interact with a screen of the surround vision system.

The controller may be configured to over-ride the command device, for example when an output from the anxiety monitoring device exceeds a first threshold value or falls below a second threshold value.

Preferably, the controller is configured to record the scene displayed, the commands given via the command device and the output of the or each anxiety monitoring device, where the apparatus includes anxiety monitoring devices. Such information may be useful to a psychologist or therapist analyzing the effect of the apparatus.

The apparatus may include a library of sets of scenes, each set of different a theme or a different level of challenge for the same theme. Such sets of scenes would be developed in conjunction with trained psychologists, therapists or researchers. By developing such sets of scenes, the therapy may then be delivered by less highly qualified persons, who are trained in use of the apparatus.

Preferably, the library includes scenes or sets of scenes that do not relate to the theme, and instead are scenes that the individual receiving therapy would find relaxing.

The invention also provides a method of conducting cognitive behavior therapy using an apparatus comprising:
a surround vision system including a plurality of screens;
sets of recorded scenes each set relating to the same theme, each set representing a different level of difficulty to the individual receiving therapy in dealing with the theme;
a controller for controlling the displaying of sets of scenes on the screens of the surround vision system; and
a command device for use by a person overseeing the delivery of therapy to the individual or the individual receiving therapy for controlling the display of scenes on the screens of the surround vision system;
displaying selected sets of scenes in a selected order on the screens of the surround vision apparatus.

The method may include the further step of monitoring anxiety levels in the person receiving the therapy.

The method may include the further step of selecting sets of scenes to be displayed on the screens of the surround vision apparatus according to the monitored anxiety levels of the person receiving the therapy.

The method may include the further step of overriding the scenes selected for display by the command device according to the monitored anxiety level. If the monitored anxiety level exceeds a threshold level.

The method may include the step of interrupting the display of scenes relating to the theme for which the individual is receiving treatment and displaying scenes that the individual would find relaxing. The method may also include the step of displaying such relaxing scenes prior to displaying scenes relating to the theme in order to relax the individual prior to beginning treatment, for example for a period of a few minutes, typically approximately 5 minutes

The apparatus and method of the invention has been found to be very effective in helping individuals with autism spectrum disorder overcome phobias, and in becoming accustomed to difficult social situations.

### BRIEF DESCRIPTION OF THE DRAWINGS

In the drawings, which illustrate preferred embodiments of the apparatus for use in the performance of cognitive behavior therapy of the invention:

FIG. 1 is a schematic representation of apparatus according to the invention;

FIG. 2 is a schematic illustration of a camera used in the generation of images for projection into the apparatus illustrated in FIG. 1; and

FIG. 3a is a schematic representation of the apparatus in use;

FIG. 3b is a side view of the apparatus illustrated in FIG. 3a;

FIG. 3c is a front view of the apparatus illustrated in FIG. 3b; and

FIG. 3d is a view of a viewing screen.

### DETAILED DESCRIPTION

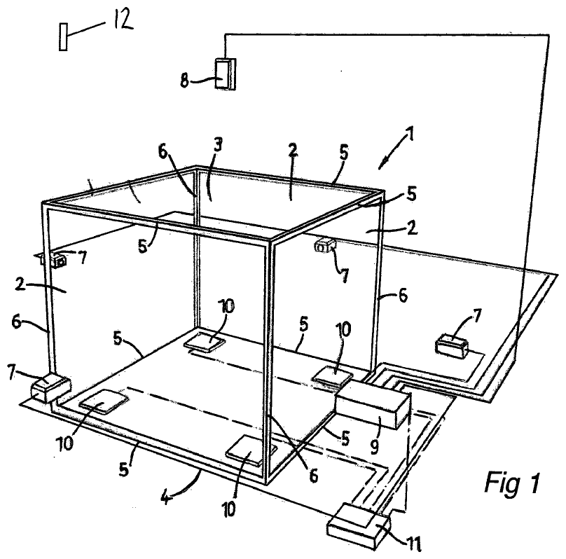

Referring now to FIG. 1, there is shown apparatus including an enclosure 1 comprising four projection screens 2 forming the walls of the enclosure 1, a fifth projection screen 3 constituting the enclosure's ceiling, and a floor 4. Each projection screen 2 is attached to horizontal and vertical frame members 5, 6. The projection screen 3 is attached to the upper horizontal frame member 5, whilst the floor 4 is attached to the lower horizontal frame members 5. The result is a completely enclosed space. At least one of the screens 2 is either moveable or includes a closable access, thereby permitting a person to enter the enclosure 1.

In this example, four video projectors 7 are arranged outside the enclosure 1 and are aligned to project images onto the screens 2. A fifth video projector 8 is located above the screen 3 and is aligned to project images onto the screen 3. Each projector 7, 8 projects an image onto one of the screens 2, 3, respective projected images each filling one of the screens 2, 3.

As an alternative, the screens 2, 3 and projectors 7, 8 may both be replaced by flat panel display or screen technology, such as plasma or LCD screens.

The projection of images onto the screens 2, 3 is controlled by a controller in the form of one or more processors (in the example one processor 9), which process the video and audio streams.

The floor 4 of the room 1 includes loud speakers 10, one in each corner of the floor 4. The delivery of sound to the loudspeakers 10 is controlled by the processor 9, and is fed through an amplifier 11 to ensure adequate loudness within the room 1.

The processor 9 synchronizes the transmission of images to the projectors 7, 8. In one method of synchronization the processor identifies time stamps in the frames of recorded data. If the image projected by one of the projectors is more than 1/100.sup.th second out of synchronization with the images projected from the other projectors, the processor executes a synchronization routine (in this case image frames are transmitted at 1/100.sup.th second intervals. If image frames are transmitted at different intervals, such as 1/50.sup.th second intervals, then the processor is arranged to execute its synchronization routine if the transmission of images by one of the projectors is out by more than the transmission rate, of in the latter case 1/50.sup.th second). This is important, since poor synchronization leads to a person in the enclosure 1 losing his point of reference. Rather than using the processor 9 to perform the synchronization of images projected onto the screens, the image data may be fed to the processor and hence the projectors in a synchronous stream.

Another function of the processor 9 is to synchronize the transmission of images via the projectors with the transmission of sound via the amplifier 11 and loudspeakers 10. The processor is adapted to control the transmission of sound, and the loudness of that sound to any one of the speakers 10. This serves two purposes. First, sound can be properly associated with the images projected on the screens 2, 3, for example where the image is of a vehicle the sound and loudness of that sound from any of the speakers 10 changes so that the sound appears to emanate from the vehicle, where-ever it is on the screens 2, 3. Second, where interactivity is provided (described in greater detail below), the processor 9 can direct sound at a desired loudness to one or more of the screens on which images are projected as a result of an interactive act.

A command device in the form of a remote control 12 is configured to communicate with the processor 9 in order to determine the images projected on the screens 2, 3.

The apparatus may include an anxiety monitoring device, which may be arranged to communicate with the processor 9. The scenes may have information associated with them indicative of level of anxiety likely to be caused. The processor 9 may be configured to override an input of the command device where the scene that would be projected on to the screens is indicated as causing anxiety significantly different from a threshold level sensed by the anxiety monitoring device.

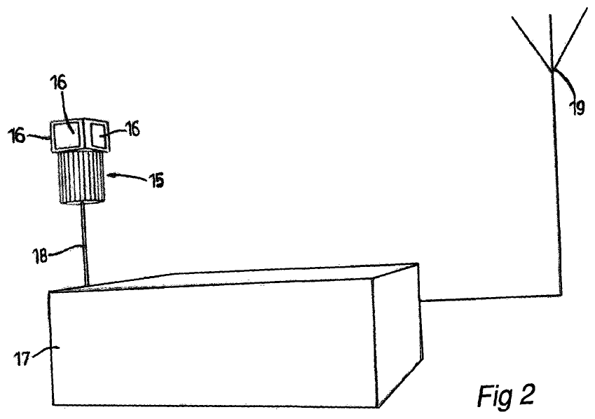

FIG. 2 illustrates a digital camera 15 comprising four camera heads 16 (two heads being fully visible in FIG. 2). Digital signals from the camera heads 16 are transmitted to a digital video recorder 17 by connection 18. The digital video recorder 17 may retransmit the recorded signals via a transmission antenna 19. Each camera head 16 includes a lens, and a digital video processor. A compression unit may also be included, but is not essential. Signals produced by the digital video processors may be transmitted to the digital video recorder either by cable, or a radio signal. Where transmission is by a radio signal, the camera is provided with a common processor that receives output signals from the digital video processors. The common processor converts the digital video signals into a radio signal and transmits this to the video recorder 17. Where a cable is used, a common processor is not required, the digital video signals being transmitted directly from the digital video processors to the video recorder.

The images projected via projectors 7, 8 may be computer generated images or images recorded using a camera, for example a camera of the type illustrated in FIG. 2.

The system provides one immersive image extending 360 degrees around the screens 2. To achieve this, the field of view of each camera head 16 must be at least 90 degrees (the lens shooting footage for the ceiling screen needs to shoot at not less than 121 degrees). The desired image can be achieved either by matching the image taken by the camera to the shape of the screen 2 on to which it is to be projected, or by processing the images to fit the screens. In order to remove aberrations and distortions advanced image processing techniques may be employed such as correction for alignment distortions, perspective distortions, pincushion distortions, barrel distortions. Whereas one camera may be employed for each screen, equally a number of cameras may be employed to capture the scene to be projected onto any one screen, with the images from each camera being stitched together to provide an apparently seamless image for projection onto a screen.

The images projected onto the screens may be interactive. To provide for interactivity, it s necessary to include in the apparatus certain input devices, such as gyroscopic sensors, touch sensitive screens, movement sensors for sensing movement of people within the enclosure.

In use, the person receiving therapy is located in the room 1. A therapist selects the images to be displayed on the screens 2, 3, and using the remote control 12 may perform the usual commands that are associated with the projection of images, i.e. play, fast forward, rewind, select from an image library, etc. The therapist may be situated in the room 1 or outside the room 1. The apparatus may include communication means to permit communication between the therapist and the person receiving therapy where the therapist is situated outside the room 1. The remote control may be comprised in a tablet computer for example.

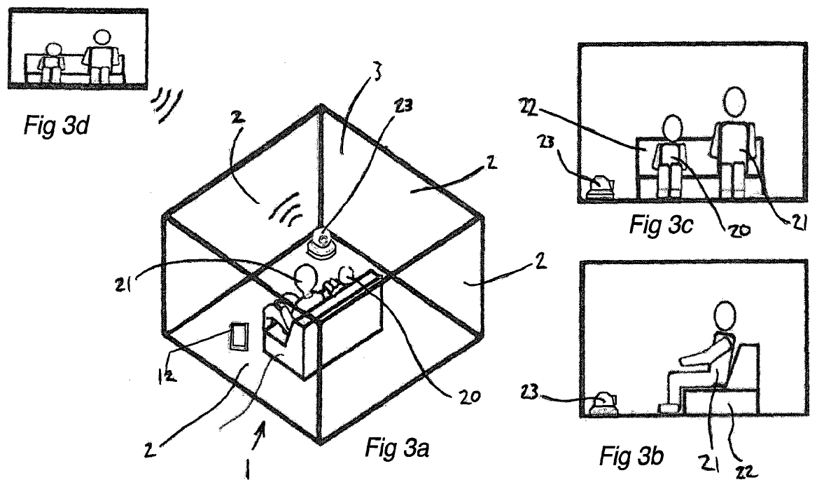

FIGS. 3a to 3d best illustrate use of the apparatus. In FIGS. 3a to 3b it can be seen that the patient 20 and the therapist 21 are both seated on a sofa 22 within the enclosure 1. The enclosure 1 is as described with reference to FIG. 1. The remote control may be used either by the patient 20 or the therapist 21, as directed by the therapist.

It can be seen that a camera 23 is located in the enclosure 1. The purpose of the camera is to capture the behaviour of the patient and therapist during therapy. The camera 23 is a web cam in the present example.

FIG. 3d illustrates a screen 24 such as a visual display unit. The pictures from the camera 23 are displayed live on the screen 24. In the example, the screen 24 is situated in a room separate from the enclosure 1. One purpose of the screen 24 is to allow observation of the therapy within the enclosure 1 to be observed by the patient's parents or carers. Another purpose of the screen 24 is to allow researchers and other professionals to observe the behavior of the patient and therapist during therapy.

### EXAMPLES

The apparatus and method of the invention were used with five children as described in detail below:

Each child received four 20-30 minute sessions in the apparatus. Two sessions took place during one visit with a 15 minute break in the middle. A therapist was in the room with the child the whole time. The therapist regularly checked in with the child about their level of anxiety on the visual scale and also coached them in relaxation techniques and coping self-talk.

Relaxing scenes were generic and included a dolphin scene, a field scene with relaxing, swaying grass and a colorful balls scene where balls pile up and then crash to the ground. Participants were allowed to choose which scene they wanted.

### Participant 1

12 year old with diagnosis of Asperger's syndrome and Dyspraxia.

Issue: unable to cross bridges, particularly if water underneath

Scene: Recreation of the Armstrong Bridge in Jesmond. Scene was developed which allowed the water level underneath the virtual bridge to be gradually increased and also to increase the height of the bridge in three increments.

After four sessions the participant lost their fear of crossing bridges. In real life he is now able to cross any bridge. He is also now able to do rock climbing and to be able to climb stairs e.g. in a multi story car park.

### Participant 2

12 year old with diagnosis of autism spectrum disorder.

Issue: phobia of pigeons

Scene: playground scene where the number of pigeons was gradually increased, both flying in and taking off

After four sessions able to do many things he couldn't previously—sit by a window in his house, walk past pigeons at entrance to shopping mall, no longer has panic attacks.

### Participant 3

8 year old with a diagnosis of Asperger's syndrome.

Issue: Following a minor car accident where his grandmother was driving, the participant had developed a phobia of being a passenger in a car when a woman was driving.

Scene: A virtual car was developed, which looked very similar to his grandmothers car. He virtually got in the car with the therapist and together they drove through a city scene.

After two sessions the child had overcome his phobia and when he arrived at the Blue Room for his second visit he told us he had been out driving with both his grandmother and mother.

### Participant 4

11 year old with a diagnosis of Asperger's syndrome.

Issue: Social phobia around shopping

Scene: Petrol station kiosk where the target was to pick up a newspaper and talk with a virtual shop assistant. Gradually the amount of speaking to the shop assistant was increased.

After four sessions the participant was able to buy a newspaper in real life. He then progressed to shopping in a shopping mall with his father waiting inside the shop. Three months post Blue Room sessions he was shopping independently with friends.

### Participant 5

9 year old with a diagnosis of autism spectrum disorder.

Issue: afraid to travel on crowded buses. Parents did not drive so this caused major problems for the family if they needed to get anywhere.

Scene: Bus stop with shelter. Bus arrives and stops and the number of people on the bus is gradually increased. Participant virtually gets on bus with therapist.

After four sessions the participant was no longer afraid of crowded buses and could travel with his family without incident. This was translated to metros and he is now able to travel on crowded metros as well.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of performing cognitive behavior therapy, the method comprising:
providing a surround vision system including a plurality of screens, the surround vision system comprising:
an enclosure including at least a plurality of walls, a ceiling and a floor, the enclosure providing a completely enclosed space, and within the enclosure a position for both an individual receiving therapy and a therapist providing therapy during the provision of therapy, and a camera associated with the position within the enclosure, wherein each of the plurality of walls and the ceiling each include a respective screen, wherein one of the plurality of walls includes a closable access to and from the enclosure; and
at least one processor and either a plurality of projectors configured to project images onto the respective screens for display thereon or the respective screens are display screens configured to display images thereon, such that the images may be viewed from within the completely enclosed space, wherein the at least one processor synchronizes the display of images on each screen such that the images are perceived by the individual receiving therapy as being uninterrupted, wherein images are comprised in frames and individual frames each include a time stamp, and wherein the at least one processor synchronizes the projection of images by identifying the time stamp in each individual frame and synchronizing the time stamps of successive individual frames;
providing sets of recorded scenes each relating to the same theme, each set representing a different level of difficulty to the individual receiving therapy in dealing with the theme;
providing a controller for controlling the displaying of sets of scenes on the screens of the surround vision system;
providing a command device usable by either the therapist providing therapy or the individual receiving therapy from within the enclosure for controlling the display of scenes on the screens of the surround vision system;
directing the camera located in the enclosure towards the position for the individual receiving therapy and the therapist providing therapy for recording the behavior of said individual receiving therapy and said therapist providing therapy during use of the apparatus;
displaying selected sets of scenes in a selected order on the screens of the surround vision system; and
recording during use of the apparatus the behavior of the individual receiving therapy and the therapist providing therapy with the camera located in the enclosure directed towards the position for the individual receiving therapy and the therapist providing therapy.

2. A method according to claim 1, including the further steps of:
providing the individual receiving the therapy with one or more monitoring devices selected from the group consisting of a heart rate monitor, a blood pressure sensor, a skin temperature sensor and an eye tracker;
monitoring the individual receiving the therapy by monitoring outputs of the one or more monitoring devices;
comparing the monitored outputs with threshold levels; and
recording, by the controller, the outputs of each of the one or more devices.

3. A method according to claim 2, including the further steps of:
providing an indicative level of at least one of: heart rate, blood pressure, skin temperature and eye movement for each set of scenes; and
selecting sets of scenes to be displayed on the screens of the surround vision apparatus according to the monitored outputs of the one or more provided monitoring devices and the indicative level of at least one of: heart rate, blood pressure, skin temperature and eye movement for each set of scenes.

4. A method according to claim 3, including the further step of overriding the scenes selected for display by the command device according to the monitored anxiety level outputs of the one or more monitoring devices.

5. A method according to claim 4, wherein the step of overriding the scenes selected for display compares the monitored outputs of the one or more provided monitoring device against the indicative level of at least one of: heart rate, blood pressure, skin temperature and eye movement associated with the scenes selected for display, and overrides the scenes selected for display if the monitored anxiety level exceeds the indicative level of at least one of: heart rate, blood pressure, skin temperature and eye movement associated with the scenes selected for display.

6. A method according to claim 1, further including the step of interrupting the display of scenes relating to the theme for which the individual is receiving-therapy and displaying a set of relaxation scenes selected from the group consisting of: a dolphin scene; a field scene with swaying grass; and a scene displaying balls piling up and crashing to the ground, and wherein the individual selects the relaxation scenes using the command device.

7. A method according to claim 1, further including the step of displaying a set of relaxation scenes selected from the group consisting of: a dolphin scene; a field scene with swaying grass; and a scene displaying balls piling up and crashing to the ground, prior to displaying scenes relating to the theme.

8. An apparatus for use in the conduct of cognitive behavior therapy, the apparatus comprising:
a surround vision system comprising:
an enclosure including at least a plurality of walls, a ceiling and a floor, the enclosure providing completely enclosed space, and within the enclosure a position for both an individual receiving therapy and a therapist providing therapy during the provision of therapy, and a camera associated with the position within the enclosure, wherein each of the plurality of walls and the ceiling include a respective screen, wherein one of the plurality of walls includes a closable access to and from the enclosure; and
at least one processor and either a plurality of projectors configured to project images onto the respective screens for display thereon or the respective screens are display screens configured to display images thereon, such that the images may be viewed from within the completely enclosed space, wherein the at least one processor synchronizes the display of images onto each screen such that the images are perceived by the individual receiving therapy as being uninterrupted, wherein images are comprised in frames and individual frames each include a time stamp, and wherein the at least one processor synchronizes the projection of images by identifying the time stamp in the each individual frame and synchronizing the time stamps of successive individual frames;
sets of recorded scenes each relating to the same theme, each set representing a different level of difficulty to the individual receiving therapy in dealing with the theme;
a controller for controlling the displaying images of a set on the screens of the surround vision system; and
a command device adapted to communicate with the controller and usable either by the therapist providing therapy or by the individual receiving therapy from within the enclosure for controlling the display of scenes on the screens of the surround vision system;
wherein the camera is located in the enclosure and directed towards the position for the individual receiving therapy and the therapist providing therapy for capturing the behaviour of said individual receiving therapy and said therapist providing therapy during use of the apparatus.

9. Apparatus according to claim 8, wherein the surround vision system includes a sound system configured to play and emit sound synchronized with the images shown on the screens of the system.

10. Apparatus according to claim 8, further including one or more devices selected from the group consisting of a heart rate monitor, a blood pressure sensor, a skin temperature sensor and an eye tracker.

11. Apparatus according to claim 10, wherein the or each device is in communication with the controller.

12. Apparatus according to claim 8, wherein the command device comprises a hand held remote control.

13. Apparatus according to claim 8, wherein the command device is adapted to interact with a screen of the surround vision apparatus.

14. Apparatus according to claim 8, including an eye tracking system wherein the command device comprises a part of the eye tracking system.

15. Apparatus according to claim 8, wherein the command device includes a glove adapted to interact with a screen of the surround vision system.

16. Apparatus according to claim 10, wherein the controller is configured to override the command device when an output from the one or more devices selected from the group consisting of the heart rate monitor, the blood pressure sensor, the skin temperature sensor and the eye tracker exceeds a threshold value.

17. Apparatus according to claim 10, wherein the controller is configured to record the scene displayed, the commands given via the command device and the output of the one or more devices selected from the group consisting of the heart rate monitor, the blood pressure sensor, the skin temperature sensor and the eye tracker.

18. Apparatus according to claim 8, further including a library of sets of senes, wherein a set of scenes is one of:
a plurality of scenes each of a different scenario; and
a plurality of scenes each of a different level of challenge for the same scenario; and
a plurality of scenes each directed to relaxing the individual receiving therapy.

19. Apparatus according to claim 8, wherein the position for the individual receiving therapy and the therapist providing therapy is a seat

20. Apparatus according to claim 8, wherein the apparatus further comprises a display screen situated outside the enclosure and wherein the camera configured to capture the behavior of the patient and therapist during use of the apparatus is connected to the display screen situated outside the enclosure for display of images captured by said camera on said display screen in real time.

21. The method of claim 1, wherein the at least one processor executes a synchronization routine when the time between the image projected by one of the projectors is greater than the frame transmission rate.

22. The method of claim 1, wherein the individual image time stamps are transmitted to the at least one processor and the at least one processor outputs the images in a synchronous stream.

23. A method of performing cognitive behavior therapy, the method comprising:
providing a surround vision system including a plurality of screens, the surround vision system comprising:
an enclosure including at least a plurality of walls, a ceiling and a floor, the enclosure providing a completely enclosed space, and within the enclosure a position for both an individual receiving therapy and a therapist providing therapy during the provision of therapy, and a camera associated with the position within the enclosure, wherein each of the plurality of walls and the ceiling each include a respective screen, wherein one of the plurality of walls includes a closable access to and from the enclosure; and
at least one processor and either a plurality of projectors configured to project images onto the respective screens or the respective screens are display screens configured to display images thereon such that the images may be viewed from within the completely enclosed space, wherein the at least one processor synchronizes the projection of images onto each screen such that the images are perceived by the individual receiving therapy as being uninterrupted; and
one or more devices selected from the group consisting of a heart rate monitor, a blood pressure sensor, a skin temperature sensor and an eye tracker;
providing sets of recorded scenes each relating to the same theme, each set representing a different level of difficulty to the individual receiving therapy in dealing with the theme;
providing a controller for controlling the displaying of sets of scenes on the screens of the surround vision system;
providing a command device usable by either the therapist providing therapy or the individual receiving therapy from within the enclosure for controlling the display of scenes on the screens of the surround vision system;
directing the camera located in the enclosure towards the position for the individual receiving therapy and the therapist providing therapy for recording the behavior of said individual receiving therapy and said therapist providing therapy during use of the apparatus;
displaying selected sets of scenes in a selected order on the screens of the surround vision system; and
recording during use of the apparatus the behavior of the individual receiving therapy and the therapist providing therapy with the camera located in the enclosure directed towards the position for the individual receiving therapy and the therapist providing therapy;
wherein the controller is configured to record the scenes displayed, the commands given via the command device, and outputs of the one or more devices selected from the group consisting of the heart rate monitor, the blood pressure sensor, the skin temperature sensor and the eye tracker.
